(19) United States
(12) Patent Application Publication
(10) Pub. No.: US 2015/0202396 A1
(43) Pub. Date: Jul. 23, 2015

(54) ENDOTRACHEAL TUBE APPARATUS

(71) Applicants: Boston Scientific Scimed, Inc., Maple Grove, MN (US); Mayo Foundation for Medical Education and Research, Rochester, MN (US)

(72) Inventors: JAMES P. ROHL, Prescott, WI (US); KIMBERLY CLARK, Coon Rapids, MN (US); BRUCE FORSYTH, Hanover, MN (US); GREGORY J. SHERWOOD, North Oaks, MN (US); UDAYA B. PRAKASH, Rochester, MN (US); CRAIG E. DANIELS, Rochester, MN (US); DAVID E. MIDTHUN, Oronoco, MN (US); RODRIGO CARTIN-CEBA, Rochester, MN (US)

(21) Appl. No.: 14/601,532

(22) Filed: Jan. 21, 2015

Related U.S. Application Data
(60) Provisional application No. 61/929,756, filed on Jan. 21, 2014.

(51) Int. Cl. A61M 16/04; A61M 16/20; A61M 16/08

(52) U.S. Cl. CPC A61M 16/0486; A61M 16/0447; A61M 16/0493; A61M 16/0497; A61M 16/0816; A61M 16/208

(57) ABSTRACT

This document provides endotracheal tube designs with features that can improve the efficacy, patient safety, and efficiency of endobronchial access. For example, this document provides endotracheal tube designs with features for atraumatic passage of the tube's distal tip through the vocal folds. In addition, this document provides endotracheal tubes that have variations in strength and flexibility properties along the length of the tube in an advantageous pattern to enhance the efficacy of the endotracheal tube.

### CLAIM OF PRIORITY

This application claims priority to U.S. Provisional Application Ser. No. 61/929,756, filed on Jan. 21, 2014, the entire contents of which are hereby incorporated by reference.

### BACKGROUND

1. Technical Field

This document relates to endotracheal tube devices with features that can improve endobronchial access efficacy and efficiency.

2. Background Information

Endobronchial access is necessary for a variety of beneficial medical procedures dealing with the interior areas of the airways and lungs. Such procedures include endoscopic visual inspection, ultrasonic inspection, and taking biopsies of tissues and collection of fluids. Complications arising from endobronchial access can include sore throat, lacerations of the lips or gums, chipped teeth, vocal cord damage, laryngospasm, tracheal or bronchial perforation, nerve injury, and so on. To mitigate the risks of such injuries, an endotracheal tube is installed in a patient to provide a protective passageway for the various instruments to be inserted and removed repeatedly from the patient's bronchial area. The endotracheal tube can thereby protect the patient's tissue structures between the lips and the vocal folds.

Endotracheal tubes are also commonly used as conduits for oxygenation and ventilation. For example, endotracheal tubes are used for airway management in the settings of general anesthesia, critical care, mechanical ventilation, and emergency medicine.

Endotracheal tubes are installed by performing a tracheal intubation process. During tracheal intubation, an endotracheal tube is inserted through the patient's mouth (or sometimes through a tracheotomy) and advanced in the airway, through the vocal folds, such that the distal-most end of the endotracheal tube terminates at a location about two to six centimeters above the bifurcation of the carina.

### SUMMARY

This document provides endotracheal tube designs with features that can improve the efficacy, patient safety, and efficiency of endobronchial access. For example, this document provides endotracheal tube designs with features for atraumatic passage of the tube's distal tip through the vocal folds. In addition, some endotracheal tube embodiments provided herein have variations in strength and flexibility properties along the length of the tube in an advantageous pattern to enhance the efficacy of the endotracheal tube.

In a first general aspect, this document features an endotracheal tube device. The endotracheal tube device comprises an elongate tubular body that defines an inner working channel. The elongate tubular body has a tubular wall, and two auxiliary lumens are defined within the tubular wall. An outer shape of a transverse cross-section of at least a first portion of the elongate tubular body is an oblong shape. The endotracheal tube also comprises a reinforcing member that is embedded within the tubular wall. The endotracheal tube also comprises a connector that extends from a proximal end of the elongate tubular body. The connector is configured for attachment to a respiratory machine. The endotracheal tube also comprises a distal tip that extends from a distal end of the elongate tubular body. The distal tip has a beveled leading edge with a distal-most portion.

In various implementations of the endotracheal tube device, the two auxiliary lumens are disposed at about 180 degrees apart from each other within the tubular wall. The two auxiliary lumens are optionally disposed within the tubular wall in coincidence with the oblong cross-sectional shape of the first portion of the elongate tubular body. An outer shape of a transverse cross-section of a second portion of the elongate tubular body may be circular in some embodiments. The inner working channel may have a circular cross-sectional shape in some embodiments. Optionally, an outer size of the elongate tubular body can be tapered along a longitudinal length of the elongate tubular body. In some such embodiments, an outer size of a distal portion of the elongate tubular body may be smaller than an outer size of a proximal portion of the elongate tubular body. The outer size of a distal portion of the elongate tubular body may be smaller than the outer size of a proximal portion of the elongate tubular body by at least 1 millimeter in some embodiments. Optionally, a wall thickness of the tubular wall at a distal portion of the elongate tubular body may be thinner than a wall thickness of the tubular wall at a proximal portion of the elongate tubular body.

In various implementations of the endotracheal tube device, the endotracheal tube device may further comprise an inflatable member that is in fluid communication with at least one of the two auxiliary lumens. In some embodiments, the inflatable member can comprise a balloon cuff. In alternative embodiments, the inflatable member may comprise an inflatable elliptical mask. In some embodiments, the reinforcing member may comprise a coiled wire. A winding pitch of the coiled wire may be greater at a proximal portion of the elongate tubular body than at a distal portion of the elongate tubular body in some embodiments. The two auxiliary lumens of the endotracheal tube device are disposed at about 180 degrees apart from each other within the tubular wall in some embodiments. The distal-most portion of the distal tip may optionally be orientated angularly at about 90 degrees between the two auxiliary lumens. The two auxiliary lumens of the endotracheal tube device are disposed at about 180 degrees apart from each other within the tubular wall in some embodiments. The distal-most portion of the distal tip may optionally be orientated angularly to coincide with one of the two auxiliary lumens. In some embodiments, the elongate tubular body may further comprise an inner-most surface that is configured to be a lubricious surface.

In another aspect, this document features a method of using an endotracheal tube. The method comprises (i) inserting the endotracheal tube into a patient and (ii) orienting the beveled leading edge of the distal tip in relation to the patient's vocal folds. The orienting is performed using a self-steering characteristic of the oblong transverse cross-section of the elongate tubular body and the pre-determined orientation of the beveled leading edge in relation to two auxiliary lumens. In some embodiments, the endotracheal tube comprises a tubular wall and has two auxiliary lumens that are defined within the tubular wall. An outer shape of a transverse cross-section of at least a first portion of the elongate tubular body is an oblong shape. The endotracheal tube also comprises a reinforcing member that is embedded within the tubular wall. The endotracheal tube also comprises a connector that extends from a proximal end of the elongate tubular body. The connector of the endotracheal tube is configured for attachment to a respiratory machine. The endotracheal tube also comprises a distal tip that extends from a distal end of the elongate tubular body. The distal tip of the endotracheal tube has a beveled leading edge with a distal-most portion.

In various implementations of the method, the two auxiliary lumens of the endotracheal device used for the method may be disposed at about 180 degrees apart from each other within the tubular wall, and the distal-most portion of the distal tip may be orientated angularly at about 90 degrees between the two auxiliary lumens. In some embodiments of the endotracheal tube used for the method, the two auxiliary lumens may be disposed at about 180 degrees apart from each other within the tubular wall, and the distal-most portion of the distal tip may be orientated angularly to coincide with one of the two auxiliary lumens. Optionally, an outer size of the elongate tubular body of the endotracheal device used for the method may be tapered along a longitudinal length of the elongate tubular body such that an outer size of a distal portion of the elongate tubular body is smaller than an outer size of a proximal portion of the elongate tubular body. The elongate tubular body of the endotracheal device used for the method may optionally comprise an inner-most surface that is configured to be a lubricious surface.

In another aspect, this document features another endotracheal tube embodiment. This endotracheal tube embodiment comprises an elongate tubular body that defines an inner working channel. The elongate tubular body comprises a tubular wall, and an outer shape of a transverse cross-section of at least a first portion of the elongate tubular body is an oblong shape. The endotracheal tube also comprises a reinforcing member that is embedded within the tubular wall. The endotracheal tube also comprises a connector that extends from a proximal end of the elongate tubular body. The connector can be configured for attachment to a respiratory machine. The endotracheal tube also comprises a distal tip that extends from a distal end of the elongate tubular body. The distal tip can have a beveled leading edge with a distal-most portion.

In various implementations of the endotracheal tube embodiment, an outer size of the elongate tubular body may be tapered along a longitudinal length of the elongate tubular body. In some such embodiments, an outer size of a distal portion of the elongate tubular body may be smaller than an outer size of a proximal portion of the elongate tubular body. The outer size of a distal portion of the elongate tubular body may be smaller than the outer size of a proximal portion of the elongate tubular body by at least 1 millimeter in some embodiments. Optionally, a wall thickness of the tubular wall at a distal portion of the elongate tubular body may be thinner than a wall thickness of the tubular wall at a proximal portion of the elongate tubular body. In some embodiments, the elongate tubular body of the endotracheal tube may comprise an inner-most surface that is configured to be a lubricious surface.

Particular embodiments of the subject matter described in this document are designed to realize one or more of the following advantages. First, some embodiments of the endotracheal tubes provided herein reduce the risk of damaging the vocal folds during the intubation process. The risk of damaging the vocal folds is reduced at least in one aspect by the design of the distal tip portion that includes a beveled leading edge by which the distal tip can be gently advanced through the vocal folds. In another aspect, the risk of damaging the vocal folds during intubation is reduced by the endotracheal tube's self-steering nature by virtue of having an oblong cross-sectional shape.

Second, some embodiments of the endotracheal tubes provided herein advantageously have a variation in strength and flexibility along the length of the endotracheal tube. For example, a proximal region may have a strong crush resistant construction to provide substantial protection from patient bite forces. A distal region may have a flexible construction to facilitate atraumatic intubation. In some embodiments, such variations in strength and flexibility are accomplished using materials of different durometers and/or wall thicknesses along the length of the endotracheal tube. In some embodiments, such variations in strength and flexibility are accomplished using one or more reinforcing members of varying configurations along the length of the endotracheal tube. In some embodiments, a combination of such features are used.

Third, in some embodiments at least the distal portion of the endotracheal tube has a thin wall that provides a low profile for a given inner working channel diameter. Such a feature can advantageously provide a spacious working channel while keeping the outer diameter sized suitably for a patient's anatomy. In some embodiments, the endotracheal tubes are tapered to a smaller outer diameter at the distal portion.

Fourth, clinicians will find the insertion process of the endotracheal tubes provided herein to be more efficient and convenient than conventional endotracheal tubes because of the features such as the flexibility and self-steering properties of the distal portion.

Fifth, the endotracheal tubes provided herein can improve patient outcomes, and reduce procedural time and cost by enabling a less traumatic and less complex intubation process.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is partial cutaway side view of a patient that has been intubated using an endotracheal tube in accordance with some embodiments provided herein.

FIG. 2 is a perspective view of an example endotracheal tube in accordance with some embodiments provided herein.

FIG. 3 is a perspective view of another example endotracheal tube in accordance with some embodiments provided herein.

FIG. 4 is a perspective view of another example endotracheal tube in accordance with some embodiments provided herein.

FIGS. 5A through 5D are side view illustrations of an example endotracheal tube in accordance with some embodiments provided herein.

FIG. 6A is a longitudinal cross-sectional view of the example endotracheal tube of FIGS. 5A through 5D.

FIGS. 6B and 6C are magnified illustrations of portions of the longitudinal cross-sectional view of FIG. 6A.

FIG. 7A is a perspective view of the endotracheal tube of FIGS. 5A through 5D.

FIG. 7B is a perspective view of an example reinforcing member that can be used with the endotracheal tube of FIG. 7A.

FIG. 8A is side view of the example endotracheal tube of FIGS. 5A through 5D.

FIGS. 8B through 8D are illustrations of transverse cross-sections at various locations along the length of the endotracheal tube of FIG. 7A.

FIGS. 9 and 10 are illustrations of alternative transverse cross-sections of endotracheal tubes in accordance with some embodiments provided herein.

Like reference numbers represent corresponding parts throughout.

### DETAILED DESCRIPTION

This document provides endotracheal tube (“ET tube”) designs with features that can improve the efficacy, patient safety, and efficiency of endobronchial access. For example, this document provides ET tube designs with features for atraumatic passage of the tube's distal tip through the vocal folds. In addition, some ET tube embodiments provided herein have variations in strength and flexibility properties along the length of the tube in advantageous patterns to enhance the efficacy of the ET tube. Further, in some embodiments the ET tubes provided herein are tapered so that the distal portion of the ET tube has a smaller outer diameter than a proximal portion of the tube. Such a feature can advantageously provide a spacious working channel while keeping the outer diameter sized suitably for a patient's anatomy, and while also providing a more flexible and atraumatic distal portion.

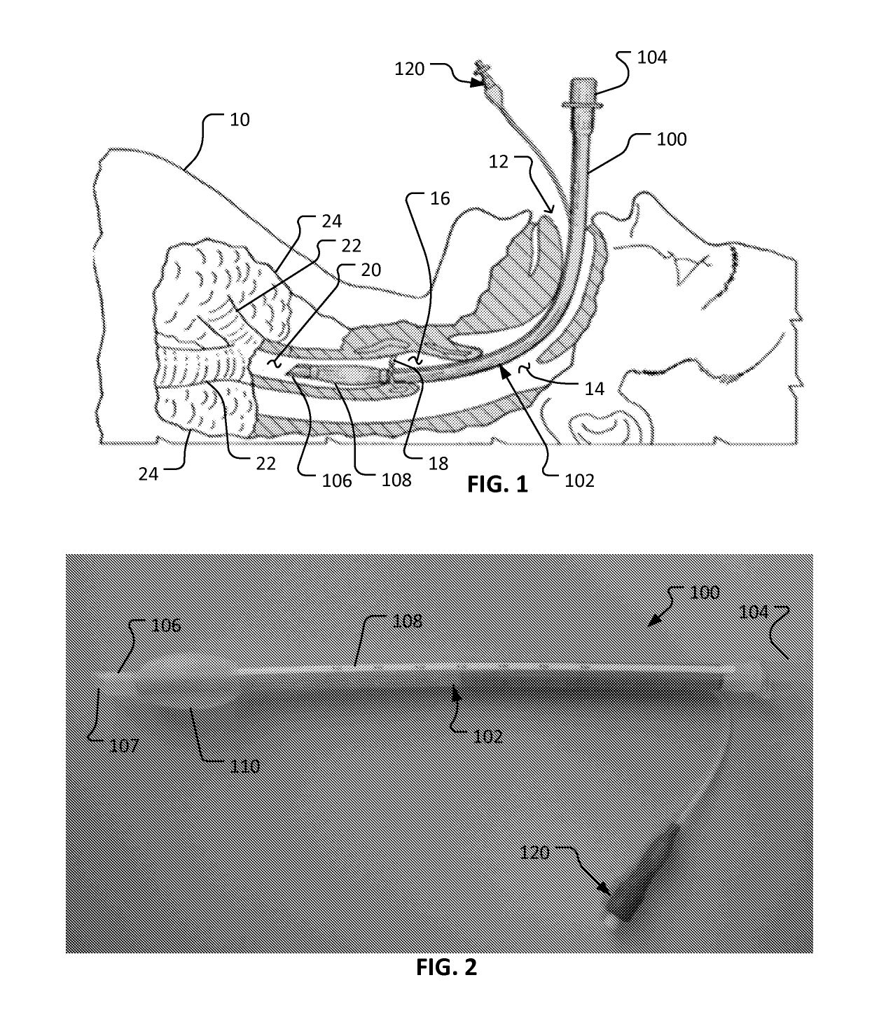

Referring to FIG. 1, an endobronchial access procedure can be performed on a patient 10 using the ET tube embodiments provided herein. In this example, the patient 10 has been intubated through the patient's mouth 12 to install an example ET tube 100. In alternative examples, the intubation may take place through the patient's nose or through a tracheotomy. The example ET tube 100 and other embodiments of ET tubes provided herein are also suitable for such alternative intubation techniques.

During the intubation process, the ET tube 100 is passed through the mouth 12, and into the patient's pharynx 14. As shown, the ET tube 100 is bent or radiused into a curved configuration as it passes from the mouth 12 and into the pharynx 14. As the intubation process progresses, the ET tube 100 passes from the pharynx 14 and into the patient's larynx 16 (voice box region). The patient's vocal folds 18 are in the larynx 16. The ET tube 100 is carefully passed through the delicate vocal folds 18 and into the patient's trachea 20. A distal tip 106 of the ET tube 100 is finally positioned in the trachea 20 just a few centimeters short of the bifurcation of the primary bronchi 22, which are branches to the lungs 24.

The passage of the distal tip 106 of the ET tube 100 through the vocal folds 18 presents a risk of injury to the vocal folds 18. In some situations, the vocal folds 18 may not be fully open when the ET tube 100 is inserted. Further, when the ET tube 100 is placed through the vocal folds 18, it is done somewhat blindly. Consequently, the clinician installing the ET tube 100 may have a difficult time inserting the distal tip 106 through the vocal folds 18 without causing trauma to the vocal folds 18. For example, the distal tip 106 may poke, rub, stretch, or abrade the delicate vocal folds 18. As described further below, the ET tube 100, and other embodiments provided herein, have design features that facilitate atraumatic passage of the distal tip 106 through the vocal folds 18.

Referring now to FIGS. 1 and 2, the example ET tube 100 includes a tube 102, a proximal connector 104, the distal tip 106, a marking stripe 108, a balloon cuff 110, and a cuff inflation port and tube assembly 120. The proximal connector 104 is affixed to the proximal end of the tube 102. The distal tip 106 is affixed to the distal end of the tube 102. The balloon cuff 110 surrounds the tube 102 in an area near to the distal end of the tube 102. The longitudinal marking stripe 108 is visible along the side of the tube 102. The cuff inflation port and tube assembly 120 is attached to the tube 102 near to the proximal end of the tube 102. The cuff inflation port and tube assembly 120 is in fluid communication with the balloon cuff 110 via one or more auxiliary lumens in the tube 102.

The tube 102 can be made in various sizes, in terms of lengths and diameters. The various sizes of the tube 102 adapt the ET tubes provided herein for use with all patient body sizes, from pediatric to adult patients. For example, the inner diameter of the primary lumen or working channel of various embodiments of the ET tubes provided herein can range from about 2.0 millimeters to about 12 millimeters. The inner diameter of the depicted embodiment of ET tube 100 is about 9.5 millimeters. The lengths of various embodiments of the ET tubes provided herein can range from about 7 centimeters to about 35 centimeters. The length of the depicted embodiment of ET tube 100 is about 32 centimeters (from the proximal end of the tube 102 to the distal-most part of the distal tip 106).

In some embodiments, the tube 102 is tapered. That is, the outer diameter of the tube 102 is different at different locations along the longitudinal length of the tube 102 in some embodiments. In particular embodiments, the outer diameter of the tube 102 is tapered and the inner diameter of the tube 102 is also tapered. The tapers can be the same or different. In some embodiments, just one of the inner or outer diameter is tapered, while the other of the inner or outer diameter is a consistent diameter. For example, the outer diameter of the depicted embodiment of tube 102 is tapered, while the inner diameter of the tube 102 is a consistent diameter. In some such embodiments, the tapered outer diameter of the tube 102 can vary along the longitudinal length of the tube 102 by about 0.5 millimeters, about 1.0 millimeter, about 1.5 millimeters, about 2.0 millimeters, or more than 2.0 millimeters, and such variance can be any dimensional amount therebetween.

In the depicted embodiment of tube 102, the amount of taper of the outer diameter is about 1.0 millimeter. That is, the outer diameter at the proximal end portion is about 1.0 millimeter larger than the outer diameter at the distal end portion. In this example embodiment, the outer diameter of the proximal end portion of the tube 102 is about 12 millimeters, and the outer diameter of the distal end portion of the tube 102 is about 11 millimeters.

Since the outer diameter of the example tube 102 is tapered while the inner diameter of the tube 102 is a consistent diameter, the wall thickness of the tube 102 is inconsistent along the longitudinal length of the tube 102. For example, because the outer diameter of the proximal end portion of the tube 102 is about 12 millimeters and the outer diameter of the distal end portion of the tube 102 is about 11 millimeters (while the inner diameter of both the proximal and distal end portions are the same), the wall thickness at the distal end portion is therefore about 0.5 millimeters thinner than the wall thickness at the proximal end portion of the tube 102. The thinner wall can provide advantages, such as more flexibility and providing a large working channel with a corresponding relatively small outer diameter.

The tube 102 can include a primary lumen (working channel) and, in some embodiments, one or more other auxiliary lumens. For example, the tube 102 includes two other lumens in addition to the primary lumen.

The tube 102 can have various transverse cross-sectional shapes, as will be described further below. In some embodiments, the outer cross-sectional shape (profile) is a circle. In some embodiments, the outer cross-sectional shape is an oblong, such as an oval, an irregular shape, and the like. In particular embodiments, the outer cross-sectional shape is different at different locations along the longitudinal length of the tube 102. For example, as will be described further below, the transverse cross-sectional shape of the tube 102 is an oblong for most of the longitudinal length of the tube 102 except for the distal end portion, which is a circle.

From the field of mechanics, it is known that the area moment of inertia of a tube's cross-sectional shape characterizes the tube's ability to resist bending (flexure). The higher the area moment of inertia, the more resistant the tube is to bending.

When the cross-sectional shape of a tube is circular, the area moment of inertia is the same in all planes of bending. However, when the cross-sectional shape of the tube is an oblong, the tube will have different area moments of inertia in different planes of bending. For example, considering an elliptical cross-sectional area as shown below (where ‘a’ is the major radius and ‘b’ is the minor radius), the area moment of inertia about the x axis is (π/4)ab³, whereas the moment of inertia about they axis is (π/4)ba³.

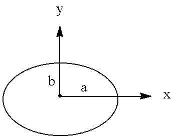

Since a>b, the area moment of inertia about they axis is greater than the area moment of inertia about the x axis. Therefore, a tube with an elliptical cross-section as shown above will bend more easily about the x axis than about they axis.

In general, a tube with an elliptical (or oblong) cross-section will tend to be more resistant to bending about the minor axis than about the major axis. For that reason, when a tube with an oblong cross-section is subject to bending, the tube will tend to self-steer (twist) so that the bending is taking place about the major axis, rather than the minor axis. As will be discussed further below, in some embodiments of the ET tubes provided herein this principle is used advantageously to orient the distal tip 106 as desired in relation to the vocal folds 18. For example, in this manner the distal tip 106 can be presented to the vocal folds 18 in an orientation that results in an atraumatic insertion of the distal tip 106 through the vocal folds 18, even though the insertion may be performed somewhat blindly.

The tube 102 can be made from various polymeric materials. For example, in some embodiments the tube 102 is made of materials including, but not limited to, polyvinyl chloride (PVC), plastisol, silicone rubber, latex rubber, polyethylene, polyurethane, fluoroelastomers, flouropolymers, and the like, and combinations thereof.

Materials of different durometers may be included for the construction of some embodiments of tube 102. For example, in some embodiments, the distal tip may have a higher durometer than other portions of the tube 102. Further, the use of materials with different durometers at different locations along the length of the tube 102 can be used selectively to make some portions of the tube 102 more flexible than other portions.

In some embodiments, the tube 102 is constructed from multiple layers of tubing. Multi-layered tubes 102 may include two, three, four, five, six, or more than six layers of construction. The various layers may be included to create a tube 102 with advantageous properties. For example, an inner-most liner layer that has a particularly lubricious inner surface may be included. The lubricious nature of the inner-most layer may facilitate the insertion of instruments, such as a bronchoscope, into and through the working channel of the tube 102 with a reduced amount of frictional resistance.

The individual tubes that make up a multi-layered tube construct can be made using various techniques. For example, techniques such as, but not limited to, extrusion, overmolding, spray molding, or dip molding can be used to make tubes, and/or to overlay an outer tubing layer onto an inner tube.

In some embodiments, a reinforcing layer can be included between the polymeric tubing layers. The reinforcing layer can add kink resistance so that the lumen will remain patent when the tube 102 is bent. Also, the reinforcing layer can increase the crush resistance of the tube 102. For example, increased crush resistance can be advantageous in the region of the tube 102 that is in the patient's mouth because, in some situations, the patient that has been intubated may bite onto the tube 102. The crush resistance from the reinforcing layer can prevent the force of the bite from negatively affecting the functionality of the tube 102, such as by partially occluding the tube 102. The reinforcing layer can be a metallic material (e.g., stainless steel) in the form of a coil, braided tube, stent-frame construction (expanded metal), and the like.

In some embodiments, the reinforcing layer is consistent along the longitudinal length of the tube 102. In some embodiments, two or more discrete reinforcing elements are each included in the tube 102 to form the reinforcing layer. The example tube 102 includes a stainless steel coil reinforcing layer. As will be described further below, the coil has a winding pitch (number of coils per unit length) that is greater in some areas along the longitudinal length of the tube 102 as compared to other areas along the tube 102 that have fewer coils per unit length. In some embodiments, the reinforcing layer can be comprised of radiopaque materials. In one such non-limiting example, the reinforcing layer can be made of a drawn stainless steel tubular wire that is filled with a noble metal such as platinum, iridium, palladium, and the like.

Still referring to FIGS. 1 and 2, the example ET tube 100 also includes a proximal connector 104. The proximal connector 104 is configured to connect with a breathing system. In some embodiments, the outer diameter of the proximal connector 104 is about 15 millimeters. The proximal connector 104 can be an injection molded thermoplastic such as, but not limited to, polycarbonate, PVC, and the like. In some embodiments, the proximal connector 104 is bonded to the tube 102. In some such embodiments, the bonding may be accomplished by solvent bonding, adhesive bonding, a UV curable adhesive, ultrasonic welding, a compression fitting, and the like.

The example ET tube 100 also includes a distal tip 106. In some embodiments, the distal tip 106 is injection molded in conjunction with the molding of a layer of the tube 102. In some embodiments, the distal tip 106 is bonded onto the end of the tube 102. In particular embodiments, one or more tubular layers of the tube 102 are bonded in the inner diameter of the distal tip 106.

The distal tip 106 can be made of biocompatible polymeric materials including, but not limited to, silicone, PVC, polycarbonate, and the like. The hardness of the distal tip 106 can be selected as desired, and may be varied from one embodiment of the ET tube 100 to another. In the depicted embodiment, the distal tip 106 is made of silicone that is about 60 or about 80 durometer. In other embodiments, the distal tip 106 may have a durometer that is lower than 60 or higher than 80.

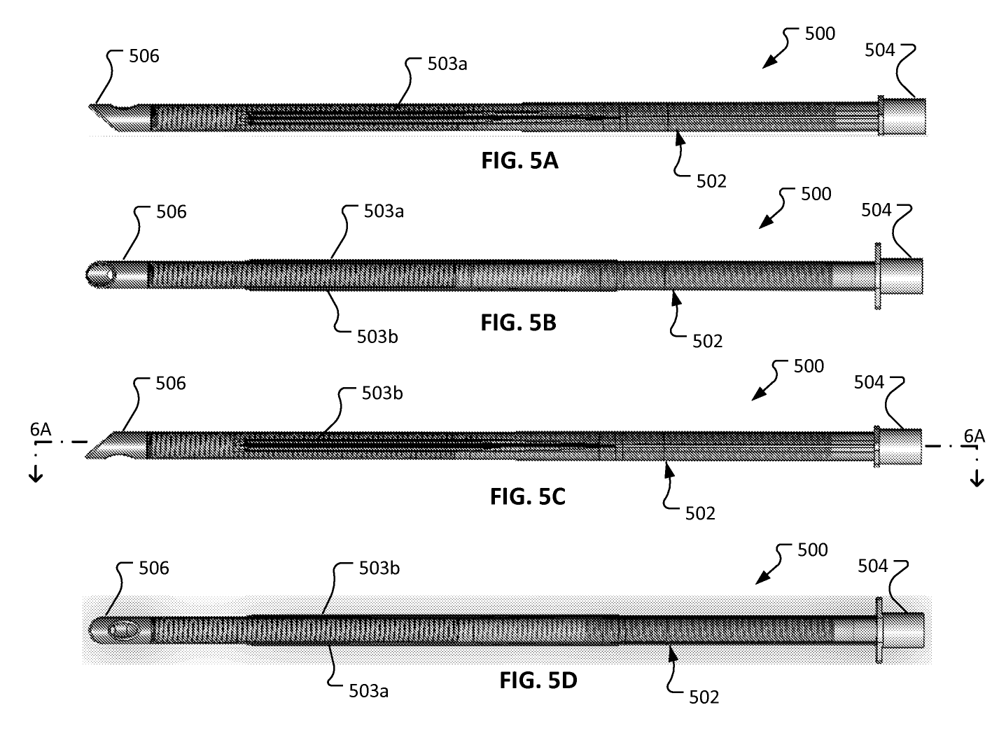

In some embodiments, the distal tip 106 may include a Murphy eye (e.g., refer to FIGS. 5A-5D). A Murphy eye is an opening in the side or wall of the distal tip 106 which allows airflow into the patient in the event that the opening at the distal end of the distal tip 106 is obstructed because of lying against the tracheal wall or being obstructed in other ways.

The distal tip 106 may also include beveled leading edge 107. While in this example, the leading edge 107 of the distal tip 106 has a single bevel, in other embodiments a double bevel, or other shapes and types of leading edges such as frustoconical, curved, radiused, and the like, and combinations thereof, may be included.

In some embodiments, the beveled leading edge 107 of the distal tip 106 is oriented in a known relationship to the oblong cross-sectional shape of the tube 102. The use of such a known relationship can advantageously facilitate the presentation the leading edge 107 to the vocal folds 18 in a desired orientation. For example, FIG. 1 shows the ET tube 100 having been curved on a radius within the region of the pharynx 14. As described above, because the tube 102 has an oblong cross-section, the tube 102 will tend to twist when subjected to bending so that the bending is taking place about the major axis, rather than the minor axis. Therefore, as the tube 102 is being inserted through the region of the pharynx 14, and as the tube 102 is being subjected to bending forces, the tube 102 will tend to twist so that the bending will take place about the major axis of the oblong cross-section. When the beveled leading edge 107 is oriented in a known relationship to the oblong cross-sectional shape of the tube 102, such twisting of the tube 102 can thereby present the beveled leading edge 107 to the vocal folds 18 in a particular pre-determined orientation as desired. The desired orientation may be such that the insertion of the distal tip 106 through the vocal folds 18 is performed in an atraumatic manner.

The example ET tube 100 also includes a longitudinal marking stripe 108. In some embodiments, two or more longitudinal marking stripes 108 are included on the tube 102. The longitudinal marking stripe 108 can provide markings that are useful to the clinician performing the intubation. Such markings can include, but are not limited to, depth markings, indications of the size of the ET tube 100, orientation markings, warnings, concise instructions, and the like, and combinations of such markings.

In some embodiments, the markings on the longitudinal marking stripe 108 are printed with biocompatible ink. In the depicted example embodiment, the markings on the longitudinal marking stripe 108 are created by laser marking. While some polymeric materials can be laser marked effectively, not all can. In some embodiments that use polymeric materials that are not suited for laser marking, an additive material that is suitable for laser marking can supplement the polymeric material. For example, in the depicted embodiment that is constructed primarily of silicone, an additive material of about 2% TiO2 was included with the material used to form a portion of the ET tube 100. As will be described further below, the TiO2 is added to the silicone material that form the auxiliary (side) lumens. The TiO2 not only provides a material that can be laser marked, the TiO2 also provides a white background that allows the laser markings to the distinctly visible on the tube 102.

Still referring to FIGS. 1 and 2, the example ET tube 100 also includes a balloon cuff 110. The cuff 110 is an inflatable region at the distal end portion of the tube 102. FIG. 1 shows the cuff 110 prior to inflation, and FIG. 2 shows the cuff 110 in an inflated configuration.

The cuff 110 forms a seal against the tracheal wall. This seal prevents gases from leaking past the cuff 110 and allows positive pressure ventilation. The seal also prevents matter such as regurgitated gastric contents going into the trachea.

In the example embodiment, the cuff 110 is made of silicone. But materials such as, but not limited to, latex, fluoroelastomers, polyurethane, polyethylene terephthalate (PET), and the like, are used in some embodiments.

The cuff 110 can be made to have a variety of different shapes when inflated. The example cuff 110 is generally cylindrical. However, in other embodiments cuff shapes such as spherical, frustoconical, and the like can be used. In some embodiments, two or more cuffs 110 are included on the tube 102.

As will be explained further below, the cuff 110 can be in fluid communication with one or more auxiliary lumens (other than the working channel) that extend along the longitudinal length of the tube 102. An inflation media, such as air, can be introduced into the one or more auxiliary lumens at a proximal end of such one or more lumens.

The example ET tube 100 also includes a cuff inflation port and tube assembly 120. The cuff inflation port and tube assembly 120 is in fluid communication with the cuff 110 via the one or more inflation media lumens. The cuff inflation port and tube assembly 120 provides a connection site for a source of inflation media that can be injected to inflate the cuff 110. For example, in the depicted embodiment a syringe can be coupled to the cuff inflation port and tube assembly 120, and the syringe can be used to inject an inflation media (e.g., air) into the assembly 120 to inflate or deflate the cuff 110. The cuff inflation port and tube assembly 120 includes a one-way valve, such that the cuff 110 can remain inflated after the syringe is removed from the assembly 120. In some embodiments, the cuff inflation port and tube assembly 120 includes a pilot balloon that provides an indication of the amount of inflation media pressure that has been received by the cuff 110.

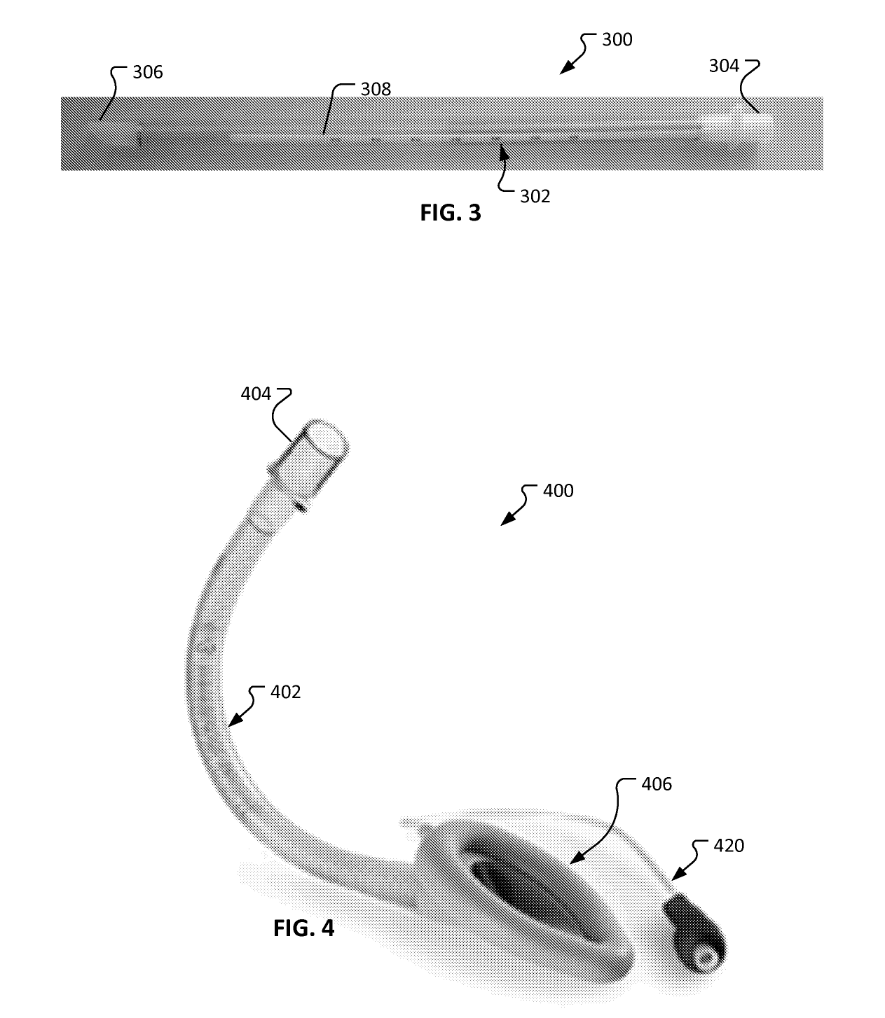

Referring now to FIG. 3, another example ET tube 300 in accordance with some embodiments is provided. The ET tube 300 includes a tube 302, a proximal connector 304, a distal tip 306, and a longitudinal marking stripe 308. This ET tube 300 embodiment is an example of a cuff-less ET tube embodiment. The tube 302, connector 304, distal tip 306, and marking stripe 308 are analogous to like components of ET tube 100 described above.

Referring now to FIG. 4, another example ET tube 400 in accordance with some embodiments is provided. The distal portion of ET tube 400 includes an inflatable elliptical mask 406 (shown here in the inflated configuration). The inflatable elliptical mask is designed to sit in the patient's hypopharynx and cover the supraglottic structures, thereby allowing relative isolation of the trachea. Hence, this type of ET tube 400 is also referred to as a laryngeal mask airway (LMA).

The LMA 400 is constructed of a tube 402 that is attached at a proximal end to a proximal connector 404, and to the inflatable elliptical mask 406 on the distal end of the tube 402. A cuff inflation port and tube assembly 420 is also included that is in fluid communication with the cuff of the inflatable elliptical mask 406. While the cuff inflation port and tube assembly 420 in this example embodiment is not integral with the tube 402, in other embodiments the tube of the cuff inflation port and tube assembly 420 is integrated with the tube 402 (e.g., like that of ET tube 100 of FIGS. 1 and 2). In some such embodiments, the cuff inflation port and tube assembly 420 can be connected to a proximal end of one or more inflation media lumens that extend along the longitudinal length of the tube 402. In particular embodiments, the one or more inflation media lumens may be positioned on an outer periphery of the working channel of tube 402, as will be described further below. The tube 402, the proximal connector 404, and the cuff inflation port and tube assembly 420 are analogous to like components of ET tube 100 described above.

FIGS. 5A through 5D illustrate four different side views of an example ET tube 500 in accordance with some embodiments provided herein. The ET tube 500 includes a tube 502, a proximal connector 504, a distal tip 506, and two auxiliary lumens 503a and 503b. The proximal connector 504 is attached to the proximal end of the tube 502. The distal tip 506 is attached to the distal end of the tube 502. The auxiliary lumens 503a and 503b are attached to the periphery of tube 502 and extend longitudinally from the proximal end of the tube 502 to the distal portion of the tube 502, but not necessarily all the way to the distal end terminus of the tube 502.

The tube 502, the proximal connector 504, and the distal tip 506 are analogous to like components of ET tube 100 described above. While this ET tube 500 does not include a cuff or an inflatable elliptical mask, it should be understood that the ET tube 500 is designed to accommodate a cuff or an inflatable elliptical mask in some embodiments. In particular embodiments, one or both of the auxiliary lumens 503a and 503b can be used as a conduit to convey an inflation media to the cuff or the inflatable elliptical mask.

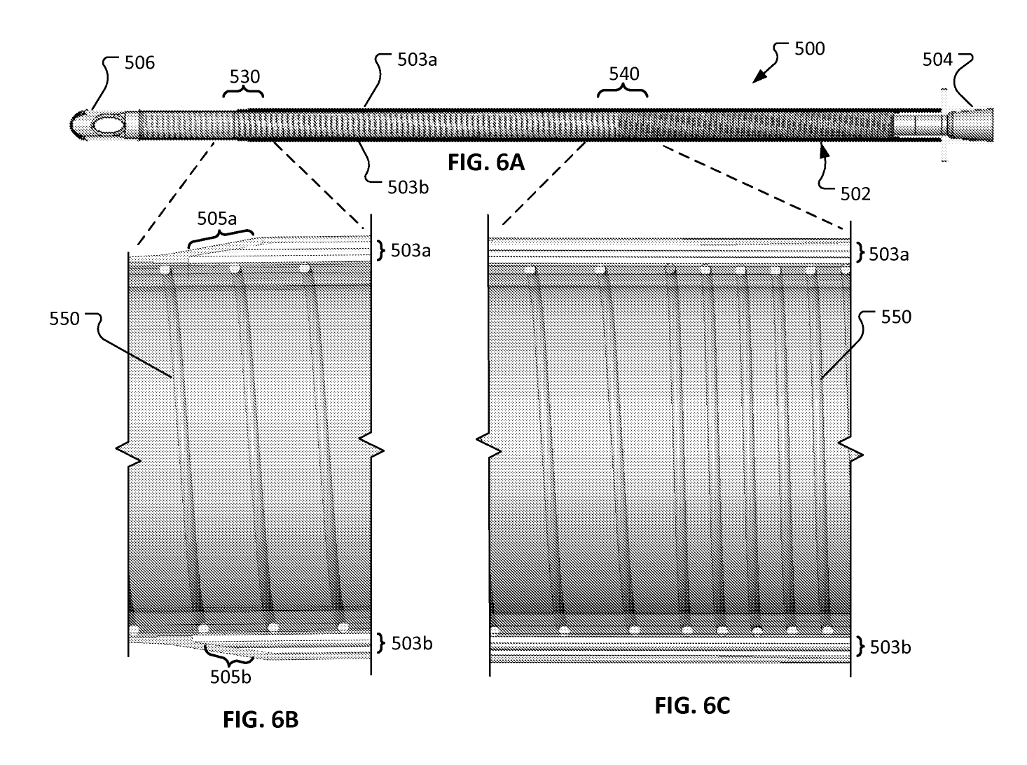

FIG. 6A is a longitudinal cross-section of ET tube 500 taken along section 6A-6A (as shown in FIG. 5C). FIGS. 6A and 6B are enlarged views of tube portions 530 and 540 respectively. In the depicted embodiment of ET tube 500, the auxiliary lumens 503a and 503b are located in the wall of tube 502 and are disposed 180 degrees apart from each other. Therefore, the cross-sectional view of FIG. 6A reveals the inner space of auxiliary lumens 503a and 503b (best seen in the enlarged FIGS. 6B and 6C).

The portion 530 of tube 502 includes the distal end portions of the auxiliary lumens 503a and 503b in this embodiment. In other embodiments, the distal end portions of the auxiliary lumens 503a and 503b can extend further towards the distal end of the tube 502 (including all the way to the distal end terminus of the tube 502 in some embodiments) or, in other embodiments, not as far distally as shown. In some embodiments, one of the auxiliary lumens 503a or 503b extends further than the other of the auxiliary lumens 503a or 503b.

The auxiliary lumens 503a and 503b include distal openings 505a and 505b respectively. In embodiments of ET tube 500 that include a cuff or an inflatable elliptical mask, the openings 505a and/or 505b can be within the interior space of the cuff or the inflatable elliptical mask. Accordingly, the auxiliary lumens 503a and/or 503b can be used as a conduit to supply an inflation media to the cuff or the inflatable elliptical mask. In some embodiments, one or both of the auxiliary lumens 503a and 503b can be used for other purposes such as, but not limited to, irrigation, sampling, administration of pharmacological agents, suction, and the like, or combinations thereof.

The portion 540 also includes the auxiliary lumens 503a and 503b. Further, within the portion 540, a reinforcing member 550 visibly transitions from a fine pitch towards the proximal portion of the tube 502, to a coarser pitch towards the distal portion of the tube 502. In this embodiment, the reinforcing member 550 is a stainless steel wire coil that is embedded within the wall of the tubing 502. In alternative embodiments, other types of reinforcing members can be used, including but not limited to, braided metallic tubes, stent-frame members, and the like, and combinations thereof. While in this example embodiment the reinforcing member 550 is one continuous coil with a varying pitch, in other embodiments two or more discreet reinforcing members can be included in a single tube construct.

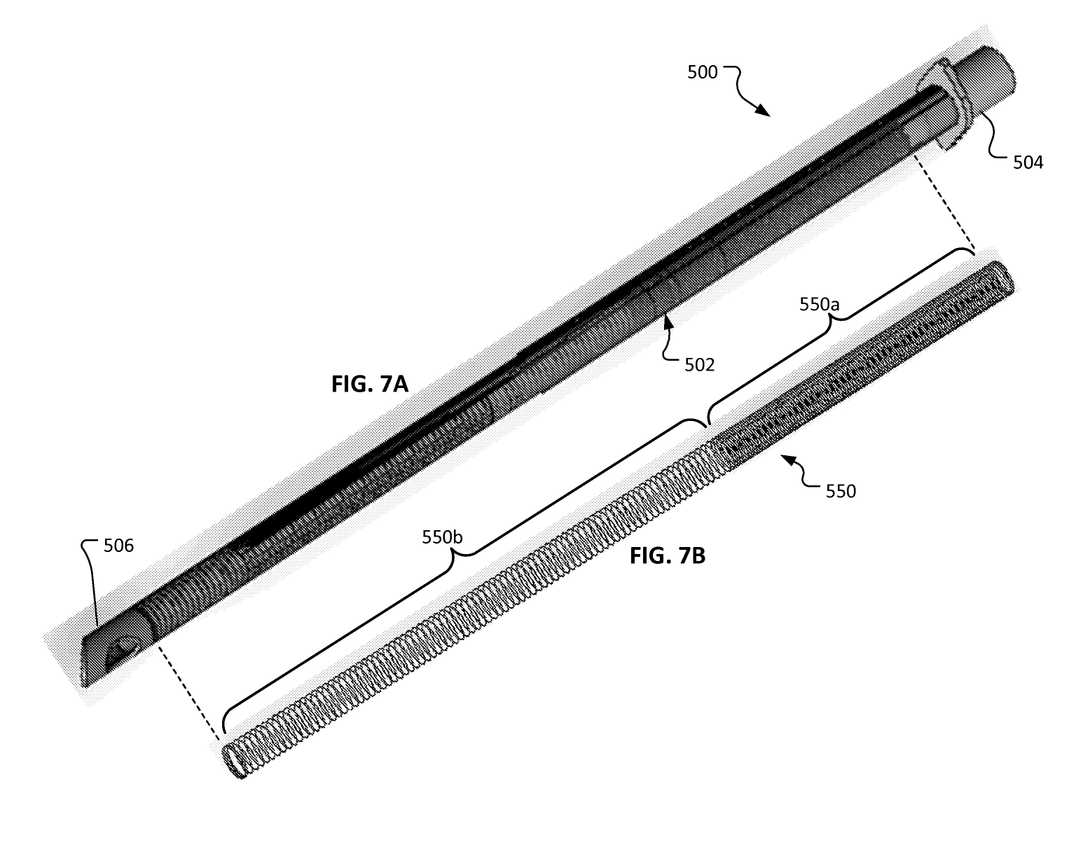

FIGS. 7A and 7B further illustrate the reinforcing member 550 in relation to the tube 502 of the example ET tube 500. In this embodiment, the reinforcing member 550 comprises two portions, a proximal portion 550a and a distal portion 550b. The proximal portion 550a is wound with a finer pitch than the distal portion 550b. By varying the pitch of the reinforcing member 550, the local flexibility and strength of the tube 502 can be selectively determined. That is, some portions of the tube 502 can be made to be more or less flexible than other portions of the tube 502 by varying the pitch of the reinforcing member 550. Similarly, some portions of the tube 502 can be made to have more or less crush strength and kink resistance than other portions of the tube 502 by varying the pitch of the reinforcing member 550.

In this embodiment, the proximal portion 550a is wound with a fine pitch to strengthen the proximal portion 550a so as to provide bite resistance, for example. The distal portion 550b, in contrast, is wound with a coarser pitch to make the distal portion 550b more flexible while still retaining a suitable amount of kink resistance.

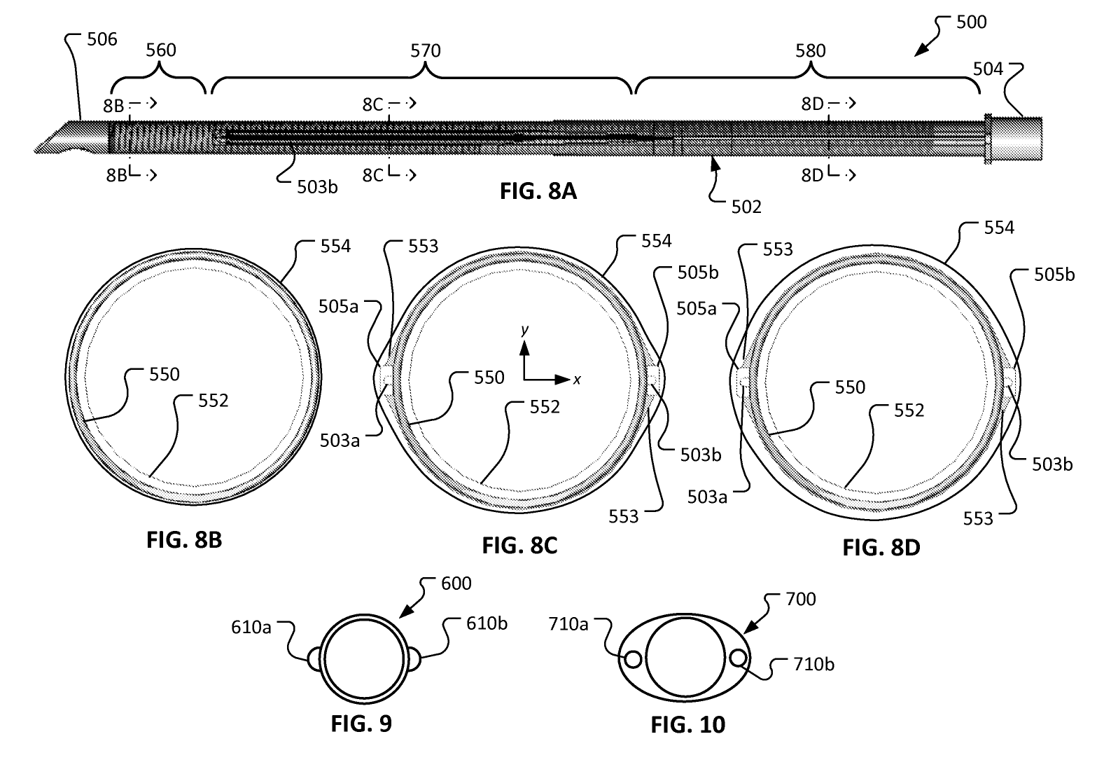

Referring now to FIG. 8A, the example ET tube 500 is shown in a side view. FIGS. 8B through 8D illustrate transverse cross-sectional views of the tube 502 at sections 8B-8B, 8C-8C, and 8D-8D respectively.

In FIG. 8A, the tube 502 is identified as having three portions, a distal portion 560, an intermediate portion 570, and a proximal portion 580. The tubes of alternative embodiments may have fewer than or more than three such portions. Also in some embodiments, the longitudinal lengths and relative position of such portions may be different than that of the ET tube 500.

In the depicted embodiment, the tube 502 is tapered. That is, the outer diameter of the tube 502 is different at different locations along the longitudinal length of the tube 502. The tubes of alternative embodiments may have a consistent outer diameter, or may be tapered in a different pattern than the tube 502 described here.

The proximal portion 580 has an outer diameter that is consistent and that is the largest of the tube portions 560, 570, and 580. The distal portion 560 has an outer diameter that is consistent and that is the smallest of the tube portions 560, 570, and 580. The intermediate portion 570 has an outer diameter that is variable (tapered). The outer diameter of the proximal end of the intermediate portion 570 matches the outer diameter of the proximal portion 580. The outer diameter of the distal end of the intermediate portion 570 matches the outer diameter of the distal portion 560. In some embodiments, the taper of the intermediate portion 570 is a constant rate of taper (a consistent angle). In other embodiments, the taper can be greater at some parts of the intermediate portion 570 than at other parts of the intermediate portion 570. In other words, the taper along the intermediate portion 570 (or other portions of the tube 502) can be inconsistent in some embodiments.

In this embodiment, the inner diameter of the tube 502 is a consistent size. The tubes of alternative embodiments may have an inner diameter that is inconsistent (e.g., tapered) along the length of the tube.

In the depicted embodiment, the distal portion 560 has a consistent outer diameter of about 11 millimeters. As described above, a range of such outer diameter sizes are envisioned so as to provide ET tube embodiments for all sizes of patients. While the depicted ET tube 500 does not include a cuff or an inflatable elliptical mask, it should be understood that the ET tube 500 is adaptable to include a cuff or an inflatable elliptical mask in some embodiments. In embodiments that include a cuff or an inflatable elliptical mask, the cuff or inflatable elliptical mask can be located at the distal portion 560 or as a substitute for the distal portion 560.

FIG. 8B is a cross-sectional view of tube 502 taken at section 8B-8B in the distal portion 560. The cross-section at 8B-8B includes an inner tube 552, a reinforcing member 550 and an outer tube 554. The reinforcing member 550 is between the inner tube 552 and the outer tube 554. Together, the inner tube 552, reinforcing member 550, and the outer tube 554 make up an integral tubing construct.

In some embodiments, the distal portion 560 of the tube 502 may also include an intermediate tube 553 (not shown, but as described below in reference to FIG. 8C) that is disposed between the outer tube 554 and the reinforcing member 550.

In some embodiments, the layers of the inner tube 552, reinforcing member 550, and the outer tube 554 become integrated together using an overmolding process, and such overmolding processes may include the use of insert molding techniques. In some embodiments, the inner tube 552, reinforcing member 550, and the outer tube 554 are extruded, spray molded, bonded, or dip molded onto each other. The layers of the inner tube 552, reinforcing member 550, and the outer tube 554 may be made of the same material, or of dissimilar materials. In the depicted embodiment, a 60 durometer silicone is used for the materials of the inner tube 552 and the outer tube 554. However, the inner tube 552 may also include an inner liner that is of a different material in some embodiments. In particular embodiments, the durometer of the materials are selected so as to create desirable flexibility and strength properties at particular portions of the tube 502. In some embodiments, the distal tip 506 is molded in the same process step as the molding of the inner tube 552. In alternative embodiments, the distal tip 506 is molded separately and then bonded to the tube 502.

In some embodiments, the inner tube 552 includes a lubricious inner surface. Such a lubricious surface can conveniently facilitate the insertion of other devices (e.g., a bronchoscope) within the working channel of the tube 502 with minimal frictional resistance. In some such embodiments, the lubricious surface can be made using an inner liner of a material such as PTFE (“polytetrafluoroethylene”), Pebax®, and the like. In other embodiments, the lubricious surface can be made by treating the inner surface of the inner tube 552 using various techniques to create a low-friction surface.

The reinforcing member 550 in the depicted embodiment is a wire coil (refer to FIG. 7B). In some embodiments, the wire coil reinforcing member 550 is preformed and placed onto the inner tube 552 prior to overmolding the outer tube 554 over the reinforcing member 550 and the inner tube 552. In other embodiments, the wire coil reinforcing member 550 is wound in tension directly onto the inner tube 552. In other words, the wire of the reinforcing member 550 can be formed into a coil by winding the wire around the inner tube 552, in a manner as if the inner tube 552 is being used as a mandrel.

The outer tube 554 at distal portion 560 has a thin wall. That is, the wall of the outer tube 554 at the distal portion 560 is thinner than the wall of the outer tube 554 at the intermediate portion 570 and at the proximal portion 580. The thin wall of the outer tube 554 in the distal portion 560 can result in the outer diameter of the distal portion 560 being smaller than the other portions 570 and 580, as described above. In some embodiments, the wall thickness of the inner tube 552 is similarly thinner at the distal portion 560 than at the other portions 570 and 580. In some such embodiments, the wall thicknesses of both the inner tube 552 and the outer tube 554 are thinner at the distal portion 560 than at the other portions 570 and 580. In this manner, the outer diameter of the distal portion 560 can be smaller than the outer diameters of the other portions 570 and 580, thereby facilitating the tapering of the ET tube 500.

FIG. 8C is a cross-sectional view of tube 502 taken at section 8C-8C in the intermediate portion 570. The cross-section at 8C-8C includes the inner tube 552, the reinforcing member 550, the outer tube 554, an intermediate tube 553, and two lumen-forming members 505a and 505b. The reinforcing member 550 is between the inner tube 552 and the intermediate tube 553. The two lumen-forming members 505a and 505b, are between the intermediate tube 553 and the outer tube 554. Together, the inner tube 552, reinforcing member 550, the intermediate tube 553, the two lumen-forming members 505a and 505b, and the outer tube 554 make up an integral tubing construct.

The intermediate tube 553 can be overmolded (or extruded, spray molded, dip molded, etc.) onto the combination of the inner tube 552 and the reinforcing member 550. The intermediate tube 553 bonds with the inner tube 552 in the interstitial spaces between the coils of the reinforcing member 550. In that manner, the intermediate tube 553 captures the reinforcing member 550 between the inner tube 550 and the intermediate tube 553 as an integral construct. In some embodiments, an adhesive is also used to initially attach the reinforcing member 550 onto the inner tube 552 prior to forming the intermediate tube 553 over the reinforcing member 550 and the inner tube 552. The intermediate tube 553 can include grooves for receiving the two lumen-forming members 505a and 505b.

The two lumen-forming members 505a and 505b are elongate members, each of which has a longitudinal channel that defines the auxiliary lumens 503a and 503b, respectively, after placement onto the intermediate tube 553. In some embodiments, the two lumen-forming members 505a and 505b are preformed (e.g., by molding or extrusion) and then assembled into the grooves defined by the intermediate tube 553. The assembly of the two lumen-forming members 505a and 505b and the intermediate tube 553 (which also includes the reinforcing member 550 and the inner tube 552) are then overmolded by the outer tube 554 to capture the two lumen-forming members 505a and 505b in place as shown. In that manner, the two auxiliary lumens 503a and 503b can be formed. In some embodiments, core members (e.g., wires or strings) are temporarily placed within the auxiliary lumens 503a and 503b during the molding of the outer tube 554. After the molding of the outer tube 554, the core members are removed. In this manner, the lumens 503a and 503b can be kept clear of material during the process step of overmolding the outer tube 554.

FIG. 8C shows the oblong cross-sectional shape created by the placement of the two lumen-forming members 505a and 505b approximately 180 degrees apart from each other within the wall of the tube 502. Consequently, as described above, the area moment of inertia about they axis is greater than the area moment of inertia about the x axis. Therefore, the tube 502, having the oblong cross-section created by the placement of the two lumen-forming members 505a and 505b as shown, will bend more easily about the x axis than about they axis. This property will, in turn, thereby facilitate a self-steering characteristic of the ET tube 500 when the ET tube 500 is subjected to bending (flexure), such as when the ET tube 500 passes from the mouth 12 and into the pharynx 14 during intubation (refer to FIG. 1). In some embodiments, this principle is used advantageously to orient the distal tip 506 as desired in relation to the vocal folds. For example, in this manner the distal tip 506 can be presented to the vocal folds in an orientation that results in an atraumatic insertion of the distal tip 506 through the vocal folds, even though the insertion may be performed somewhat blindly.

In the depicted embodiment, the distal-most portion of the distal tip 506 is oriented at 90 degrees between the two lumen-forming members 505a and 505b. In other embodiments, the distal-most portion of the distal tip 506 is oriented in alignment with one of the two lumen-forming members 505a and 505b. In still other embodiments, the distal-most portion of the distal tip 506 is oriented in a different spatial relationship in reference to the two lumen-forming members 505a and 505b. Therefore, it should be understood that, using the oblong cross-sectional shape of the tube 502 and the known orientation of the distal tip 506 in relation to the oblong cross-sectional shape, when the tube 502 is subjected to bending the tube 502 will tend to self-steer (e.g., twist) such that the distal tip 506 is presented to the vocal folds in a pre-determined orientation.

The two lumen-forming members 505a and 505b can also be conveniently used as marking stripes (e.g., refer to FIGS. 2 and 3). For example, in this embodiment a TiO2 material is added to the silicone resin material that is used to make the two lumen-forming members 505a and 505b. In some embodiments, the TiO2 makes up about 2% by weight of the material that is used for making the two lumen-forming members 505a and 505b. In other embodiments, other additive materials, and other percentages of TiO2 or other additive materials, can be similarly used. The TiO2 creates a white background color and makes the two lumen-forming members 505a and 505b suitable for laser marking. The laser marking can be performed either prior to overmolding the outer tube 554, or after overmolding the outer tube 554, or both.

FIG. 8D is a cross-sectional view of tube 502 taken at section 8D-8D in the intermediate portion 580. The cross-section at 8D-8D includes the inner tube 552, the reinforcing member 550, the outer tube 554, the intermediate tube 553, and the two lumen-forming members 505a and 505b. The reinforcing member 550 is between the inner tube 552 and the intermediate tube 553. The two lumen-forming members 505a and 505b, are between the intermediate tube 553 and the outer tube 554. Together, the inner tube 552, reinforcing member 550, the intermediate tube 553, the two lumen-forming members 505a and 505b, and the outer tube 554 make up an integral tubing construct.

In the proximal portion 580, the wall thickness of the outer tube 554 is greater than at the distal portion 560 and at much, or all, of the intermediate portion 570. In addition, the winding pitch of the reinforcing member 550 is greater (tighter) in the proximal portion 580 as compared to the distal portion 560 and the intermediate portion 570. Therefore, the strength of the tube 502 is the highest at the proximal portion 580, and the flexibility of the tube 502 is the stiffest at the proximal portion 580.

In some embodiments, the proximal portion 580 may include an attachment location for a cuff inflation port and tube assembly (e.g., refer to the cuff inflation port and tube assembly 120 of FIG. 2).

FIGS. 9 and 10 provide alternative transverse cross-sectional shapes 600 and 700 respectively. The cross-sectional shapes 600 and 700 are alternative oblong configurations. As such, the cross-sectional shapes 600 and 700 will exhibit a self-steering characteristic as described above. These and other such oblong cross-sectional shapes are envisioned within the scope of this disclosure.

The cross-sectional shape 600 is an example of a shape that is oblong while not necessarily including auxiliary lumens within the protruding side portions 610a and 610b. The cross-sectional shape 700 is an example of a shape that is oblong and that includes auxiliary lumens 710a and 710b. However, in other embodiments, the cross-sectional shape 700 may be used for ET tubes while not including one or both auxiliary lumens 710a and 710b.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. An endotracheal tube comprising:
an elongate tubular body that defines an inner working channel, the elongate tubular body comprising a tubular wall and having two auxiliary lumens that are defined within the tubular wall, wherein an outer shape of a transverse cross-section of at least a first portion of the elongate tubular body is an oblong shape;
a reinforcing member that is embedded within the tubular wall;
a connector that extends from a proximal end of the elongate tubular body, wherein the connector is configured for attachment to a respiratory machine; and
a distal tip that extends from a distal end of the elongate tubular body, wherein the distal tip has a beveled leading edge with a distal-most portion.

2. The endotracheal tube of claim 1, wherein the two auxiliary lumens are disposed at about 180 degrees apart from each other within the tubular wall such that the two auxiliary lumens are disposed within the tubular wall in coincidence with the oblong cross-sectional shape of the first portion of the elongate tubular body.

3. The endotracheal tube of claim 2, wherein an outer shape of a transverse cross-section of a second portion of the elongate tubular body is circular.

4. The endotracheal tube of claim 1, wherein an outer size of the elongate tubular body is tapered along a longitudinal length of the elongate tubular body.

5. The endotracheal tube of claim 4, wherein an outer size of a distal portion of the elongate tubular body is smaller than an outer size of a proximal portion of the elongate tubular body.

6. The endotracheal tube of claim 5, wherein the outer size of a distal portion of the elongate tubular body is smaller than the outer size of a proximal portion of the elongate tubular body by at least 1 millimeter.

7. The endotracheal tube of claim 5, wherein a wall thickness of the tubular wall at a distal portion of the elongate tubular body is thinner than a wall thickness of the tubular wall at a proximal portion of the elongate tubular body.

8. The endotracheal tube of claim 1, further comprising an inflatable member that is in fluid communication with at least one of the two auxiliary lumens.

9. The endotracheal tube of claim 8, wherein the inflatable member comprises a balloon cuff.

10. The endotracheal tube of claim 8, wherein the inflatable member comprises an inflatable elliptical mask.

11. The endotracheal tube of claim 1, wherein the reinforcing member comprises a coiled wire.

12. The endotracheal tube of claim 11, where a winding pitch of the coiled wire is greater at a proximal portion of the elongate tubular body than at a distal portion of the elongate tubular body.

13. The endotracheal tube of claim 1, wherein the two auxiliary lumens are disposed at about 180 degrees apart from each other within the tubular wall, and wherein the distal-most portion of the distal tip is orientated angularly at about 90 degrees between the two auxiliary lumens.

14. The endotracheal tube of claim 1, wherein the two auxiliary lumens are disposed at about 180 degrees apart from each other within the tubular wall, and wherein the distal-most portion of the distal tip is orientated angularly to coincide with one of the two auxiliary lumens.

15. The endotracheal tube of claim 1, wherein the elongate tubular body comprises an inner-most surface that is configured to be a lubricious surface.

16. A method of using an endotracheal tube, the method comprising:
inserting the endotracheal tube into a patient, the endotracheal tube comprising:
an elongate tubular body that defines an inner working channel, the elongate tubular body comprising a tubular wall and having two auxiliary lumens that are defined within the tubular wall, wherein an outer shape of a transverse cross-section of at least a first portion of the elongate tubular body is an oblong;
a reinforcing member that is embedded within the tubular wall;
a connector that extends from a proximal end of the elongate tubular body, wherein the connector is configured for attachment to a respiratory machine; and
a distal tip that extends from a distal end of the elongate tubular body, wherein the distal tip has a beveled leading edge with a distal-most portion, wherein the beveled leading edge is in a pre-determined orientation in relation to the two auxiliary lumens; and
orienting the beveled leading edge of the distal tip in relation to the patient's vocal folds using a self-steering characteristic of the oblong transverse cross-section of the elongate tubular body and the pre-determined orientation of the beveled leading edge in relation to the two auxiliary lumens.

17. The method of claim 16, wherein the two auxiliary lumens are disposed at about 180 degrees apart from each other within the tubular wall, and wherein the distal-most portion of the distal tip is orientated angularly at about 90 degrees between the two auxiliary lumens.

18. The method of claim 16, wherein the two auxiliary lumens are disposed at about 180 degrees apart from each other within the tubular wall, and wherein the distal-most portion of the distal tip is orientated angularly to coincide with one of the two auxiliary lumens.

19. The method of claim 16, wherein an outer size of the elongate tubular body is tapered along a longitudinal length of the elongate tubular body such that an outer size of a distal portion of the elongate tubular body is smaller than an outer size of a proximal portion of the elongate tubular body.

20. The method of claim 16, wherein the elongate tubular body comprises an inner-most surface that is configured to be a lubricious surface.
